(19) United States
(12) Patent Application Publication
(10) Pub. No.: US 2004/0138553 A1
(43) Pub. Date: Jul. 15, 2004

(54) Method, apparatus, and facility for magnetic resonance imaging dual scanning

(75) Inventor: Raymond V. Damadian, Woodbury, NY (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Fonar Corporation, Melville, NY

(21) Appl. No.: 10/721,016

(22) Filed: Nov. 21, 2003

Related U.S. Application Data
(60) Provisional application No. 60/428,846, filed on Nov. 25, 2002.

(51) Int. Cl. A61B 5/055

(52) U.S. Cl. 600/410; 600/415

(57) ABSTRACT

A method, apparatus and facility for magnetic resonance imaging (MRI). The method comprises selectively directing patients to either a first magnetic resonance scanner or a second magnetic resonance scanner, so that the patient's entire body, including the torso, extremities and head can be scanned, while at the same time not requiring either scanner to have the capability to perform a full functional body scan. In accordance with another aspect of the present invention, an apparatus for magnetic resonance imaging is disclosed wherein the apparatus occupies or requires clearance space of approximately nine feet.

### CROSS-REFERENCE TO RELATED APPLICATIONS

The present application claims priority to U.S. Provisional Patent No. 60/428,846, entitled “Method, Apparatus, and Facility For Magnetic Resonance Imaging Dual Scanning,” filed on Nov. 25, 2002, the disclosure of which is incorporated by reference herein in its entirety.

### BACKGROUND OF THE INVENTION

The present invention relates to magnetic resonance imaging and more specifically to apparatuses and facilities that allow dual magnetic resonance image scanning of patients.

Nuclear magnetic resonance imaging (“MRI”) is utilized for scanning and imaging biological tissue as a diagnostic aid, and is one of the most versatile and fastest growing modalities in medical imaging. Because MRI does not use x-rays or other ionizing radiation, it offers safety advantages over techniques such as conventional ray imaging, fluoroscopy and computerized axial tomography (CAT) imaging. Furthermore, MRI allows visualization of tissues that are difficult or impossible to depict using other techniques. Moreover, magnetic resonance imaging can show abnormal tissues in contrast to surrounding normal tissues.

As part of the MRI process, the subject patient is placed in an external magnetic field. This field is created by a magnet. The magnet may include a tubular solenoid having an interior bore, the magnet being arranged to provide the required magnetic field in a working volume within the bore. The subject can be positioned within the bore so that the part of the patient's body to be imaged is disposed within the working volume. Other magnet assemblies have two spaced-apart magnet elements such as coils or poles separated by a gap, and provide the required magnetic field in a working volume located within the gap. The portion of the subject's body to be imaged is positioned in the gap and in the working volume. The magnetic field produced by the magnet is applied to the subject tissue, and the resulting nuclear magnetic resonance (“NMR”) signals are received to yield NMR data. The NMR data is then processed using conventional techniques to produce an image of the tissue in that portion of the subject's body disposed within the working volume.

The elements of a conventional “full body” magnetic resonance scanner are sized and arranged to image any portion of a normal adult human body. Thus, the magnet is dimensioned so that the diameter of the interior bore, or the size of the gap, is large enough that the largest part of the body—the torso—will fit into the bore or gap, and hence any other part of the body will also fit into the bore or gap. The magnet and associated subject-positioning equipment are arranged so that position of the subject relative to the magnet can be varied to bring different portions of the subject's body into the working volume and thus allow scanning of essentially any part of the subject's body. In many conventional full body scanners, referred to herein as “horizontal full body scanners, n the subject to be scanned must be oriented with the long axis of his or her body in a horizontal position. Typically, the subject lies on a bed and the bed moves horizontally to position the desired portion of the subject's body in the working volume. Another type of scanner, referred to herein as “vertical full body scanners,n permit scanning of a patient while the long axis of the patient's body is in a vertical or near vertical orientation, as, for example, in a standing or sitting posture. A patient support holding a sitting or standing patient may be raised or lowered to align the desired portion of the subject with the working volume. Scanning of a subject in a vertical orientation offers significant clinical advantages. Certain vertical full body scanners, such as those in the preferred embodiments commonly assigned, copending U.S. patent application Ser. No. 09/718,946, the disclosure of which is hereby incorporated by reference herein, also allow scanning of a subject in a horizontal orientation or in intermediate orientations, and thus provide extraordinary versatility in imaging.

Full body scanners are expensive. Moreover, vertical full body scanners typically require floor to ceiling clearances of approximately eleven feet to be able to obtain an image of any portion of the body with the patient in a standing position. The required clearance is greater than the normal floor-to-ceiling clearance available in many buildings. This increases the cost of installing such a scanner in an existing building. Even where a new building is to be constructed to house a scanner, the need for such clearances adds to the cost of the building.

In addition, the fringe field effect causes the magnetic field to extend over a larger three-dimensional volume thereby affecting the space required. Thus, even horizontal field magnets require large clearance spaces.

In many cases, the capabilities of a full body scanner are not necessary. That is, it is quite often the case that the image needed is that of an extremity, limb or head and not the torso. As such, conventional scanners are often used inefficiently resulting in delayed access for those patients really requiring their use.

Smaller magnetic resonance scanning devices, referred to herein as “extremity scanners” have also been developed. These devices are designed such that the dimensions of the magnet bore or gap accommodate an extremity such as an arm or leg or the head, but do not accommodate the torso of a normal human subject.

While extremity scanners provide advantages over conventional full body scanners in terms of the space they occupy and the cost associated with their installation, an extremity scanner also suffers its own drawbacks. In particular, an extremity scanner is clearly not capable of scanning a patient's torso. As such, extremity scanners have not been as well received in the medical community as initially forecasted because of their limited functionality.

On the other hand, although a full body scanner typically can perform all the imaging functions of an extremity scanner, the conventional full body scanner has also not captured as large a market as initially thought because of cost and space concerns.

Although full body scanners may be used to scan extremities in addition to torsos, the capabilities of full body scanners are typically employed where full body scans are expected to be performed or where general imaging is required. Nevertheless, the facilities available for scanning almost always employ full body scanners. The full body scanners in such facilities typically serve a large geographic area or population. The throughput and efficiency associated with the use of the scanners at these facilities is usually far from optimal as full body scanners are typically used in large part for extremity and head scans. This results in avoidable delay for those patients who truly require the use of a full body scanner.

Thus, additional methods and facilities that provide more efficient scanning would be desirable. Scanners with lower costs would also be desirable.

### SUMMARY OF THE INVENTION

One aspect of the present invention provides a method of magnetic resonance imaging. In a method according to this aspect of the invention, a patient received at a facility is selectively directed to a first magnetic resonance scanner to scan the patient's torso or to a second magnetic resonance scanner to scan the patient's extremity or head. In this way, the two scanners in combination provide full body scanning functionality while overcoming the prior art disadvantages associated with the inefficient use of scanners. Specifically, a facility equipped with both a scanner capable of scanning the torso, such as a full body scanner, and an extremity scanner, allows for selective use of the scanner most appropriate for the anatomical area of interest. This results in an increased throughput and efficiency at such dual-scanner equipped facilities without incurring the additional expenses associated with having two full body conventional scanners.

Further in accordance with aspect of the present invention the method further comprises selectively directing subsequently received patients to the first magnetic resonance scanner or to the second magnetic resonance scanner.

The method may further desirably comprise receiving another patient at the facility and simultaneously scanning the received patient in the first magnetic resonance scanner and another patient in the second magnetic resonance scanner.

Further in accordance with this aspect of the present invention the method may further desirably comprise receiving another patient at the facility and simultaneously scanning the received patient in the second magnetic resonance scanner and another received patient in the first magnetic resonance scanner.

It may also prove further desirable to orient the received patients such that the received patient is in a recumbent position and place the received patient's head in the second scanner and scan the received patient's head.

The method may further desirably comprise executing on a processor a method comprising maintaining a list of the received patients in a queue, accessing the maintained list, and processing the list so as to selectively direct the received patients to preferably either the first or second magnetic resonance scanner.

Another aspect of the present invention provides a method for magnetic resonance imaging comprising selecting a first patient to be scanned at a facility, the facility having a first magnetic resonance imaging scanner and a second magnetic resonance imaging scanner, the first scanner being large enough to allow at least a torso of a patient to be scanned and the second scanner being large enough to allow only an extremity or the head of a patient to be scanned; scanning said first patient's torso using the first imaging scanner; selecting a second patient to be scanned at the facility; and scanning the extremity or head of said second patient using the second scanner. In accordance with this aspect of the present invention, the operation of scanning patients may be done more efficiently using both scanners.

Further in accordance with this aspect of the present invention the method for magnetic resonance imaging further desirably comprises performing scanning substantially simultaneously.

In yet another aspect of the present invention, a facility for performing magnetic resonance imaging is provided. The facility includes a first magnetic imaging apparatus capable of imaging a patient's torso and an extremity scanner preferably adapted to produce an image of a patient's extremity or head, and typically incapable of imaging the torso. In accordance with this aspect of the present invention, a facility provides full body scanning functionality without the associated space requirements and cost associated with the prior art. The first or torso-capable scanner used in such a facility may be a full-body scanner, such as a vertical full body scanner. Alternatively, the first, torso-capable scanner may be adapted to image the torso but may have a patient support with a range of motion less than that required to align the extremities, head or both with the working volume of the magnet. This reduces the clearance requirements associated with the first scanner and thus further reduces the cost of the facility.

Further in accordance with this aspect of the present invention, the first magnetic imaging apparatus further desirably comprises a first magnet defining a substantially horizontal first field axis and a first imaging volume surrounding the field axis. The first imaging volume preferably includes a veridical dimension in a direction transverse to the direction of the first field axis and a horizontal dimension in a direction parallel to the direction of the first field axis.

The first magnetic imaging apparatus may further desirably comprise a patient support capable of supporting a patient with the long axis of the patient's torso in a substantially vertical orientation. It is further preferable that the patient support be capable of moving a patient upwardly and downwardly so as to align a region of the patient's torso with the first imaging volume.

It is further preferable that the patient support be capable of supporting a patient in a weight bearing position, most preferably a sitting or standing position.

Further in accordance with this aspect of the present invention the second magnetic imaging apparatus preferably includes a second magnet defining a substantially second horizontal field axis and a second imaging volume surrounding said field axis. The second imaging volume desirably includes a vertical dimension in a direction transverse to the direction of the second field axis and a horizontal dimension in a direction parallel to the direction of the second field axis.

In yet another aspect of the present invention, an apparatus for magnetic resonance imaging is provided. In accordance with this aspect of the present invention, the apparatus comprises a magnet defining a working volume, also referred to as an imaging volume, and a patient support capable of supporting a human patient, with the long axis of the patient's torso in a substantially vertical orientation. The patient support desirably is movable upwardly and downwardly, but the range of motion of the patient support is preferably less than that required to align every possible portion of a standing patient with the working volume. For example, the vertical range of motion may be about 2 feet or less in either direction from a central or starting position. Magnetic resonance imaging apparatus according to this aspect of the invention may be designed and built so as to occupy significantly less vertical clearance space than a vertical full body scanner. Apparatus according to this aspect of the invention can be used in conjunction with an extremity scanner to provide, full body scanning functionality similar to that available through use of a vertical full body scanner, as in the facility discussed above.

### BRIEF DESCRIPTION OF THE DRAWINGS

FIG. 1A is a schematic block diagram of a facility in accordance with one embodiment of the present invention;

FIG. 1B is a diagrammatic sectional view of a full body scanner included in the facility of FIG. 1A;

FIG. 1C is a diagrammatic top plan view of an extremity or head scanner included in the facility of FIG. 1A;

FIG. 1D is a diagrammatic elevational view of the scanner depicted in FIG. 1C during a different phase of operation;

FIGS. 1E and 1F are views similar to FIG. 1D but depicting the scanner of FIGS. 1C and 1D in further phases of operation.

FIG. 2 is a flow chart of a method in accordance with one embodiment of the present invention;

FIG. 3 illustratively depicts an MRI apparatus in accordance with another aspect of the present invention; and

FIG. 4 illustratively depicts an MRI apparatus in accordance with another aspect of the present invention.

### DETAILED DESCRIPTION

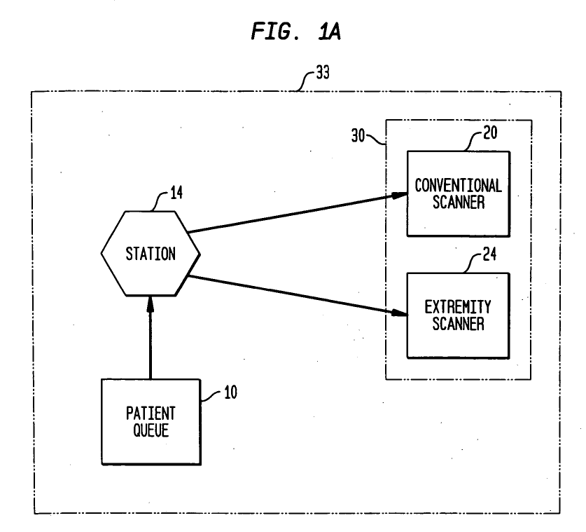

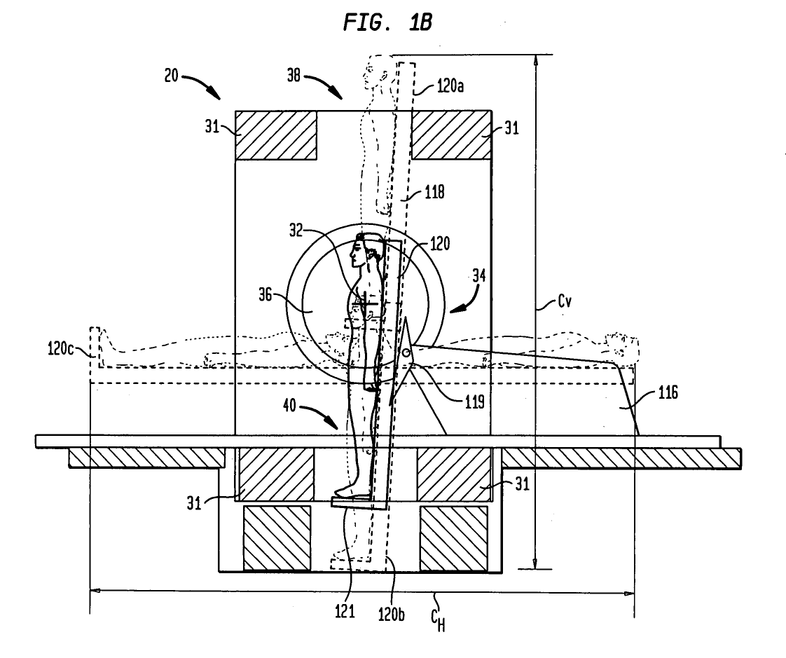

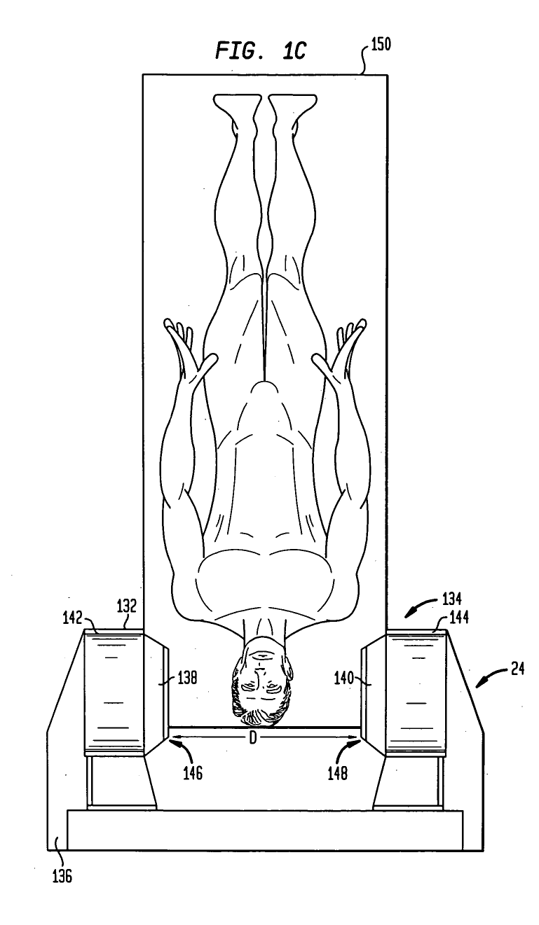

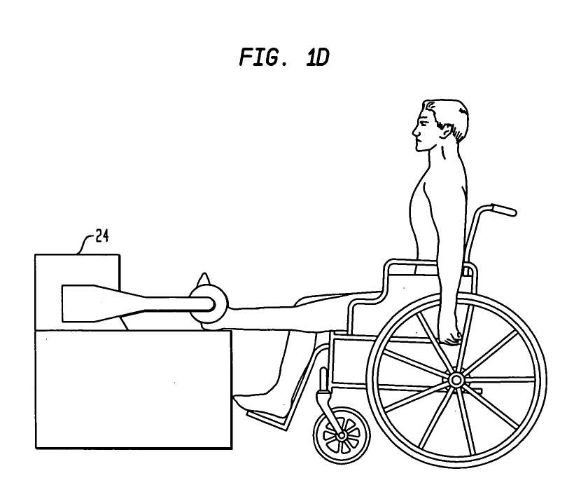

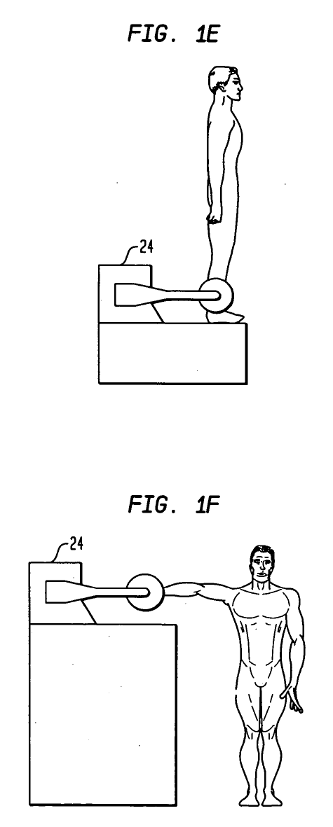

Turning now to FIG. 1A, there is depicted a plan view of a facility in accordance with an aspect of the present invention. As FIG. 1A shows, a patient from a patient queue 10, which may include one or more patients to be scanned, are directed to station 14. At station 14, the patients are then directed to a conventional full body scanner 20 or to an extremity scanner 24. In accordance with this aspect of our invention, conventional scanner 20 and extremity scanner 24 will be typically located in the same facility 30. Facility 30 may be a hospital or a doctor's office as is further discussed below.

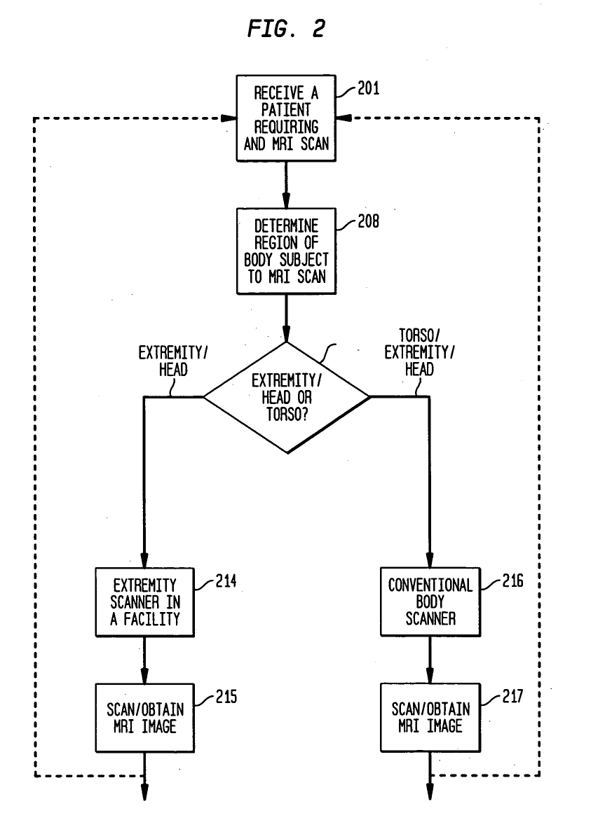

As previously noted, conventional scanner 20 is able to obtain an image of any portion of patient's entire body. Quite often, however, an entire image of the body is not needed. In such situations, extremity scanner 24 can be used to scan either the limbs or head of a patient. If the extremity scanner 24 is available and the next patient in queue requires only an extremity or head scan, then the extremity scanner is used to scan that patient's extremity or head. Conversely, if extremity scanner 24 is being used to scan another patient, the full body scanner 20 can be used to scan the next patient in queue 10 regardless of the type of scan the next patient requires. Selectively directing patients from queue 10 to scanners 20 and 24 will frequently result in the simultaneous operation of both scanners. On the other hand, where queue 10 is sparsely filled with patients, simultaneous operation of both machines may not occur. The steps involved in routing patients are schematically shown in the flow chart of FIG. 2. The process begins at step 201 when a patient requiring an MRI scan is received at, for example, station 14 of FIG. 1A. At step 208, a determination is made of whether the patient requires an extremity or torso scan. Such determination may include a diagnosis by a doctor who then refers the patient for an MRI scan. In addition or alternatively, the determination may also include a clerk or trained medical personnel review of a referral or prescription from a doctor who requested or required that the patient undergo an MRI scan. If an extremity or head scan is required, the process continues to step 214 where the extremity scanner in the facility is used to scan the patient's extremity as indicated at step 215. If, on the other hand, scanning is required of a patient's torso, the patient is directed to a conventional scanner as indicated at step 216 so that the torso of the patient may be scanned at step 217.

Alternatively, those patients who require scanning of the torso may be directed at reception station 14 to a first or full-body scan queue, whereas those patients who do not require torso scanning can be directed to a second or extremity-scan queue. Patients are directed from the full body scan queue to the full-body scanner 20, and from the extremity-scan queue to the extremity scanner 24. However, in the event that the full-body scan queue is empty or is shorter than the extremity-scan queue, patients in the extremity-scan queue are redirected to the full-body scan queue. Typically in a scanning facility first and second queues will usually be a waiting room. In most circumstances, a single waiting room may comprise both the first and second queues. However, individual waiting rooms may comprise each queue.

In accordance with this aspect of the present invention, the combination of full body scanner 20 and extremity scanner 24 improves the throughput at the facility and the operational efficiency of the medical facility. As used in this disclosure, the term “throughput” refers to the number of patients which can be scanned per unit time. In the system discussed above, patients in queue 10 may be scanned more quickly over a unit of time than in a comparable system employing only a full-body scanner, and indeed can be scanned at a throughput rate equal to or surpass that achieved by two full-body scanners. Moreover, the ability of the system to provide the appropriate scan for each patient is equal to that achieved with two full-body scanners. However, the capital investment required to construct the system is substantially less than that required for a system with two full-body scanners. In addition, efficiency is increased because those patients requiring scanning of their torso or entire body are directed to the conventional scanner 20, thereby allowing those patients in the patient queue 10 who require a conventional scanner 20 to more quickly and readily access that apparatus. In contrast, those patients in patient queue 10 that may be scanned by extremity scanner 24 are directed to that scanner. In this manner, the throughput and efficiency as measured at station 14 is increased.

Moreover, by placing conventional scanner 20 and extremity scanner 24 in the same facility, such as facility 30, the efficiency of the facility is increased without incurring the substantial additional costs associated with installing a second conventional scanner. Furthermore, the cost associated with the purchase and installation of an extremity scanner is relatively low allowing for their widespread deployment. As such, in accordance with this aspect of the invention, economies of scale may be achieved while at the same time increasing the throughput and use of MRI scanners.

In accordance with another embodiment of the present invention, patient queue 10, station 14 and facility 30 may be located in a larger facility 33. Typically, facility 33 will advantageously be a hospital or a large testing laboratory or freestanding facility. Where patient queue 10 and station 14 are not part of the same facility 33, but are instead located separately as described hereinabove, station 14 may then be located at a doctor's office. In accordance with this latter embodiment, a doctor may then selectively dispatch patients arriving at the doctor's office to an appropriate scanning facility. This may be achieved by a doctor writing a prescription for a scan. The patient would then call up a facility, perhaps recommended by a doctor or the prescribing doctor, and obtain an appointment for scanning. Alternatively, a patient may request a particular scanning facility.

As used in this disclosure, the term “facility” refers to an installation under a common management structure. The scanners and patient-directing station in a facility optionally may be physically co-located with one another, as where both of the scanners and the patient-directing station are disposed within a single physical installation, such as the campus of a medical center or within the same building. Where the scanners are physically co-located with one another, the queuing and patient-directing operations discussed above may be performed using physical patient movement. However, this is not essential. The queuing and patient-directing operations can be performed at least in part by manipulating lists or rosters of patients. For example, in another embodiment of this aspect of the present invention, station 14 may be a computer station running a software program which receives as its input, requests for scans each including the name or other data identifying a patient in queue 10 together with data identifying the scan to be performed for such patient. The data identifying the scan to be performed may identify the scan explicitly as, for example, by data denoting a “sagittal scan of the left foot” or implicitly, by data denoting a condition to be considered in planning the scan as, for example, “rule out aortic aneurism,” which implicitly denotes a need for a scan of the torso. The computer station processes the received data and selectively dispatches each patient to the appropriate scanner, such as conventional scanner 20 or extremity scanner 24. In accordance with this embodiment, station 14 may reside at a regional processing center such as Health Maintenance Organization (HMO) office or a central office of a radiology group where a request for MRI scanning is received and dispatched. Additionally, in accordance with this embodiment, station 14 may comprise any available microprocessor driven or mainframe computer having a memory for storing data and the capability to execute instructions that make up a computer program. Queue 10 may then comprise a database or plurality of databases linked to station 14 and in which resides requests for access to a scanning facility, each including patient-identifying data and data defining the type of scan to be performed. Such requests are then received and processed by the computer located at or comprising station 14 resulting in automated dispatching to an appropriate scanner residing at facility 30. The dispatching operation may consist of contacting or sending messages to each patient, or to a computer or person associated with each patient as, for example, to the referring physician's office. The message sent in respect of each patient directs the patient, or health care workers having physical charge of the patient, to physically present the patient's body at the selected scanner, and may also specify a time when the patient should appear at the selected scanner.

FIG. 1B depicts one example of a full-body scanner 20 that may be used in accordance with the above-described embodiment of the present invention. The particular full body scanner 20 of the type depicted in FIG. 1B is further described in commonly assigned U.S. patent application Ser. No. 09/718,946 and U.S. Provisional Patent Application No. 60/380,333, the disclosures of which are incorporated by reference herein. The particular scanner 20 depicted in FIG. 1B is exemplary only. In accordance with the present invention, any full body scanner may be used as the full-body scanner 20. Specifically, any horizontal or vertical full body scanner may comprise conventional scanner 20.

The full-body scanner of FIG. 1B includes a magnet frame 31 having a pair of opposed poles 30 arranged on a horizontal magnet axis 32. The opposed poles define a patient-receiving gap 34 between them. This gap has a dimension or width, in the direction perpendicular to the plane of the drawing in FIG. 1B, sufficient to accommodate a normal human torso as, for example, at least 14 inches. The magnet is arranged to provide the magnetic field required for scanning in a working volume 36 within gap 34 surrounding magnet axis 32. The magnet frame 31 has an opening 38 above the gap and a further opening 40 below the gap. A patient support assembly including an elevator having an elongated elevated frame 118 and a patient support 120 having a footrest 121 at its bottom end is also provided. Elevator frame 118 is mounted to the carriage 116 for pivoting movement about a pivot axis 119. Pivot axis 119 extends horizontally and hence is parallel to magnet axis 32. The patient support assembly includes a device for sliding patient support 120 relative to elevator frame 118. Thus, when the elevator frame and support are in a substantially vertical orientation, the patient support can move upwardly to a maximum-elevation position shown in broken lines at 120a and downwardly to a maximum-depression position 120b. In the maximum-elevation position 120a, the patient support extends about 3-6 feet above axis 32, and the bottom end of the patient support is disposed just below the axis, so that a patient standing on footrest 121 of the patient support has his or her feet disposed within the working volume 36. In the maximum-depression position 120b, the patient support extends about 3-6 feet below axis 32, so that a patient standing on the footrest 121 has his or her head disposed in the working volume. Thus, the scanner of FIG. 1B allows scanning of any part of the patient's body, including the torso, head and extremities, while the patient is in a vertical orientation. This requires a vertical clearance Cᵥ in the surrounding building structure of about 10-12 feet. The patient support is movable through a similar range of motion, between extreme position 120c shown in broken lines and the opposite extreme position shown in solid lines, while the elevator frame 118 and patient support 120 are in a generally horizontal orientation, so as to provide a similar full-body scanning capability. This requires a horizontal clearance Cₕ also on the order of 10-12 feet. Generally, by sliding the patient along the elevator and rotating the patient about the pivot axis the conventional scanner 20 allows for full body scans either in a horizontal position or a vertical position. As previously mentioned, any other full body vertical or horizontal conventional scanner may be used in accordance with this aspect of the present invention. Nevertheless, exemplary vertical full-body scanner of the type exhibited in FIG. 1B is particularly advantageous in that images of any part of the patient's body may be obtained with the patient oriented in a wide variety of patient positions which may enable better imaging and potentially better diagnosis by medical personnel. For example, the scanner of FIG. 1B allows the patient to be placed in the reverse Trendlenberg position. In addition, the scanner of FIG. 1 allows patients to enter the scanner in an upright and more natural position and advantageously reduces the feeling of claustrophobia commonly associated with MRI scanners.

Turning now to FIG. 1C, there is depicted one exemplary extremity or head scanner 24 of which can be used in the embodiment of the present invention discussed above. The extremity scanner 24 depicted in FIG. 1C is exemplary only and any other extremity scanner may suffice in accordance with this aspect of the present invention. Commonly assigned U.S. patent application Ser. No. 09/998,907, the disclosure of which is hereby incorporated by reference in its entirety, further describes the operation and construction of an extremity scanner of the type disclosed in FIG. 1C that may be used in accordance with this aspect of the present invention.

As an overview, extremity magnet 24 includes a pair of magnet assemblies 132, 134, and the frame 136, which provides supports for the first and second magnet assemblies 132, 134 and maintains a fixed distance, D, between them. The first and second magnet assemblies 132, 134 include respective first and second poles 138, 140 and respective first and second magnet enclosures 142, 144. The poles 138, 140 are disposed on opposing sides of the magnet enclosures 142, 144, such that faces 146, 148 of the poles 142, 144 are substantially parallel and facing each other. Distance D typically is about 10 to about 14 inches, i.e., too small to accommodate the torso of a normal adult human subject. A patient support 150 allows a patient to insert an extremity, e.g., a foot or a hand, or a head between magnet faces 146, 148 such that imaging of the subject extremity may be performed.

For example, a patient can lie horizontally in front of the scanner and project his or her head into the working volume for scanning as is illustratively depicted in FIG. 1C. In this instance, patient support 150 may be a stretcher. As an additional example, a patient having an ankle or knee scanned can be seated in front of the scanner and project his or her leg horizontally into the gap as is shown in FIG. 1D. Furthermore, not only can a patient extend an unsupported extremity into the working field volume of an extremity scanner, the patient can also support at least a portion of his or her weight on the subject extremity. Thus, as shown in FIG. 1E, the patient can stand between the poles, enabling a weight bearing condition that provides different and insightful information, increasing the likelihood of the successful diagnosis. In a further example, the same patient can be scanned with the extremity in a weight-bearing posture as shown in FIG. 1E and in a non-weight-bearing posture as shown in FIG. 1D, and the information recovered in the two scans can be compared, as by comparing pictorial images derived from such information, to reveal anatomical changes caused by weight bearing. Scanning of a patient's wrist or hand may be performed as exemplified in FIG. 1F. Thus, the particular extremity scanner as disclosed in the '907 application mentioned above provides extraordinary versatility.

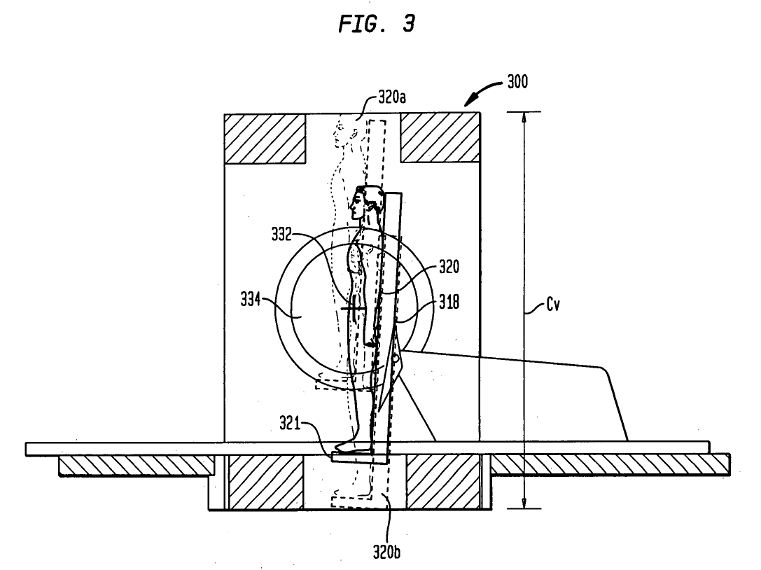

Turning now to FIG. 3, there is depicted an embodiment of a torso scanner 300 in accordance with another aspect of the present invention. Torso scanner 300 is similar to the full-body scanner 20 discussed above with reference to FIG. 1B, except that the range of motion of patient support 320 is considerably less than the range of motion of the patient support 120 in the full-body scanner. With the patient support 320 and elevator frame 318 in substantially vertical orientation, the patient support is movable between a maximum-elevation position 320a, shown in broken lines in FIG. 3, in which the patient support extends about 3 feet above magnet axis 332, and a maximum-depression position 320b, in which the patient support extends about 3 feet below magnet axis 332. For a normal human subject, vertical movement of approximately 1.5 feet up or down relative to the magnet axis 332 allows scanning of the torso. This range of motion will typically be sufficient to place any portion of the torso of a normal human subject standing on the footrest 321 at the bottom of the patient support within the imaging volume 334, and thus allows imaging of any portion of the torso with the patient in a standing, vertical posture.

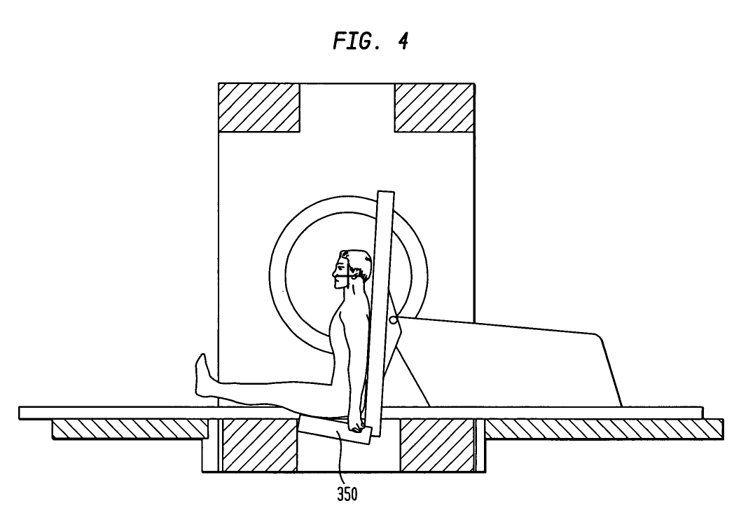

The lesser range of vertical motion in the torso scanner of FIG. 3 significantly reduces the vertical clearance Cᵥ required in the building to accommodate the scanner; the entire scanner can be accommodated in a space about 8 feet high, and hence can be accommodated in a standard-height building. This substantially reduces the cost of installation, particularly where the scanner must be installed in an existing building. It would seem at first that the reduced range of motion sacrifices some of the capability of the full-body scanner. However, where the torso scanner of FIG. 3 is used in a facility in conjunction with an extremity and head scanner such as that discussed with reference to FIGS. 1C-1E, the overall scanning capability of the facility still accommodates essentially any desired scan. Where a patient's feet must be scanned in a vertical, load-bearing posture, the patient is directed to the extremity and head scanner. Thus, in such a facility, the presence of the extremity and head scanner permits use of a slightly less capable torso scanner in place of the vertical full-body scanner without loss of scanning capability. Moreover, the torso scanner of FIG. 3 may include a seat 350 (FIG. 4) mounted to the patient support 320 so that when the seat is in place, the patient can be in a sitting posture. This positions the patient's head at a lower elevation relative to the patient support, so that the patient's head is in the imaging volume when the support is in its maximum-depression position and hence allows scanning of the head with the patient's torso in a substantially vertical orientation. The horizontal range of motion of the patient support in the torso scanner may be such as to allow full body scanning, or else may be limited in similar fashion to the vertical range of motion, so as to reduce the required horizontal clearance Cₕ. Reduction of the horizontal clearance saves floor space and hence cost in the installation, and may allow installation in facilities where an existing building does not have adequate space to accommodate the horizontal range of motion required for full body scanning.

The full-body and torso scanners discussed above with reference to FIGS. 1B and 3 are vertical scanners, in that they are capable of imaging a subject with the subject in a vertical orientation. However, using the limited range of motion required to scan only the torso of a patient may also result in a less complicated design for a horizontal scanner capable of scanning the patient only with the long axis of the body oriented in a horizontal position. In particular, the more limited range of motion required to scan only a patient's torso would reduce the horizontal clearance space required since the patient's torso can be scanned without the head or feet of the patient protruding from such scanners. For example, in such an arrangement a horizontal torso scanner can be configured to require less horizontal clearance for installation than full body horizontal scanners.

A facility including either a horizontal torso scanner or a vertical torso scanner in conjunction with an extremity scanner provides full body scanning functionality.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for magnetic resonance imaging comprising:
receiving a patient for magnetic resonance imaging at a facility, the facility having a first magnetic resonance imaging scanner and second magnetic resonance imaging scanner, the first scanner being large enough to allow at least the torso of a patient to be scanned and the second scanner being large enough to allow only an extremity or the head of a patient to be scanned; and
selectively directing said received patient to the first magnetic resonance scanner or to the second magnetic resonance scanner.

2. The method of claim 1 further comprising selectively directing subsequently received patients to the first magnetic resonance scanner or to the second magnetic resonance scanner.

3. The method of claim 1 further comprising:
receiving another patient at the facility; and
simultaneously scanning said received patient in the first magnetic resonance scanner and said another received patient in the second magnetic resonance scanner.

4. The method of claim 1 further comprising:
receiving another patient at the facility; and
simultaneously scanning said received patient in the second magnetic resonance scanner and said another received patient in the first magnetic resonance scanner.

5. The method of claim 1 further comprising:
orienting said received patient such that said received patient is in a recumbent position;
placing said received patient's head in the second scanner; and
scanning said received patient's head.

6. The method of claim 1 further comprising scanning said received patient's foot with the second scanner as said received patient is oriented in a weight bearing position in the second scanner.

7. The method of claim 1 wherein selectively directing comprises executing on a processor, maintaining a list of said received patients in a queue, accessing said maintained list, and processing said list so as to selectively direct said received patients to either the first or second magnetic resonance scanner.

8. A method for magnetic resonance imaging comprising:
selecting a first patient to be scanned at a facility, the facility having a first magnetic resonance imaging scanner and second magnetic resonance imaging scanner, the first scanner being large enough to allow at least the torso of a patient to be scanned and the second scanner being large enough to allow only an extremity or the head of a patient to be scanned;
scanning said first patient using the first imaging scanner;
selecting a second patient to be scanned at the facility; and
scanning the extremity or head of said second patient using the second scanner.

9. The method of claim 8 further comprising performing scanning substantially simultaneously.

10. A facility for performing magnetic resonance imaging, comprising:
a first magnetic resonance imaging apparatus capable of producing an image of a patient's torso; and
an extremity scanner adapted to produce an image of a patient's extremity or head.

11. The facility of claim 10, wherein said first magnetic imaging apparatus further comprises a first magnet defining a substantially horizontal first field axis and a first imaging volume surrounding said first field axis, said first imaging volume having a vertical dimension in a direction transverse to the direction of said first field axis and a horizontal dimension in a direction parallel to the direction of said first field axis.

12. The facility of claim 11, wherein said first magnetic imaging apparatus further comprises a patient support capable of supporting a patient with the long axis of the patient's torso in a substantially vertical orientation and moving the patient upwardly and downwardly so as to align a region of the patient's torso with the first imaging volume.

13. The facility of claim 12, wherein said patient support is capable of supporting a patient in a weight bearing position.

14. The facility of claim 12, wherein said patient support is capable of supporting a patient in a sitting position.

15. The facility of claim 10, wherein said second magnetic imaging apparatus includes a second magnet defining a substantially second horizontal field axis and a second imaging volume surrounding said second field axis, said second imaging volume having a vertical dimension in a direction transverse to the direction of said second field axis and a horizontal dimension in a direction parallel to the direction of said second field axis.

16. The facility of claim 14, wherein said second magnetic imaging apparatus includes a patient support capable of positioning a patient's extremity or head within the second imaging volume.

17. A magnetic imaging apparatus, comprising:
a magnet defining a substantially horizontal field axis and an imaging volume surrounding said field axis;
a patient support capable of supporting a human patient with the long axis of the patient's torso in a substantially vertical orientation; and
means for moving said patient support relative to said magnet and said imaging volume over a range of motion, said range of motion being limited to less than 1.5 feet when the patient is in said substantially vertical orientation.

18. The apparatus of claim 17, wherein the apparatus extends no more than nine feet above a surface supporting the apparatus.

19. The apparatus of claim 17 wherein said moving means comprises an elevator.

20. A combination for performing magnetic resonance imaging scanning, comprising:
an extremity scanner having a patient receiving space large enough to accommodate only an extremity or head of a patient; and
a torso scanner having a magnet defining a substantially horizontal field axis and an imaging volume surrounding said field axis, a patient support capable of supporting a human patient with the long axis of the patient's torso in a substantially vertical orientation and means for moving said patient support about said imaging volume such that a scan of the patient's torso can be obtained.
